(19) United States
(12) Patent Application Publication
(10) Pub. No.: US 2025/0000940 A1
(43) Pub. Date: Jan. 2, 2025

(54) USE OF FRUCTUS LYCII GLUCOPEPTIDE

(71) Applicants: NINGXIA QITAI TECHNOLOGY CO., LTD., Ningxia (CN); JINAN UNIVERSITY, Guangzhou, Guangdong (CN)

(72) Inventors: Rongrong HE, Guangzhou, Guangdong (CN); Wenjun DUAN, Guangzhou, Guangdong (CN); Hao GAO, Guangzhou, Guangdong (CN); Guohui SU, Hong Kong (CN); Li ZHANG, Guangzhou, Guangdong (CN); Zhexiong YU, Ningxia (CN); Ling LI, Ningxia (CN); Shan LIU, Ningxia (CN); Xiaomin LIN, Guangzhou, Guangdong (CN); Yating CHENG, Guangzhou, Guangdong (CN)

(73) Assignees: NINGXIA QITAI TECHNOLOGY CO., LTD., Ningxia (CN); JINAN UNIVERSITY, Guangzhou, Guangdong (CN)

(21) Appl. No.: 18/704,884

(22) PCT Filed: May 31, 2022
(86) PCT No.: PCT/CN2022/096395

§ 371 Date: Apr. 25, 2024

(30) Foreign Application Priority Data
Oct. 26, 2021 (CN) 202111249278.9

(51) Int. Cl. A61K 38/16; A61K 36/815; A61P 25/16

(52) U.S. Cl. CPC A61K 38/168; A61K 36/815; A61P 25/16

(57) ABSTRACT

The present invention relates to the technical field of active substance development, and especially relates to the use of Fructus lycii glucopeptide. In the present invention, the protection effect of the Fructus lycii glucopeptide on Parkinson-like dyskinesia is studied by means of using a Parkinson's disease animal model as a study object. The result shows that the Fructus lycii glucopeptide can improve the Parkinson-like dyskinesia of the model animal.

### CROSS REFERENCE TO RELATED APPLICATION

This application claims the priority to Chinese Application No. 202111249278.9 filed Oct. 26, 2021, the entire contents of which are hereby incorporated by reference.

### FIELD

The present invention relates to the technical field of active substance development, and especially relates to the use of Fructus lycii glucopeptide.

### BACKGROUND

Parkinson's disease, also known as paralysis agitans, is the second largest chronic neurodegenerative disease after Alzheimer's disease, which is pathologically characterized mainly by the loss of dopaminergic neurons in the substantia nigra pars compacta of the midbrain and the appearance of Lewy bodies in neurons (the main component is α-synuclein). Abnormal behavioral function is commonly used as an indicator for the diagnosis of Parkinson's disease in clinical, mainly including Parkinsonism such as resting tremor, bradykinesia, muscle stiffness and postural instability. With the progression and deterioration of the disease, in the late stage, a series of non-motor symptoms may occur, such as depression, anxiety, cognitive disorders, sleep disorders, olfactory disorders, constipation, spatial learning and memory impairment.

Parkinson's disease is a major problem in modern medicine, the etiology is not fully understood, and there is a lack of standardized therapy. At present, the most widely used is levodopa preparation madopar, which has been used for more than 40 years since it came into the market in 1973. However, only 5%-10% of levodopa can actually penetrate the blood-brain barrier, and the rest is distributed in the body, leading to a variety of side effects including dyskinesia and orthostatic hypotension. Monoamine oxidase B inhibitors such as selegiline combined with madopar can relatively reduce the dosage of levodopa, but after the honeymoon period, the efficacy will decline, with fluctuated symptoms and obvious side effects. Therefore, the existing western medicine therapy cannot effectively prevent the development of the disease, and because patients need lifelong medication to improve symptoms, the use of traditional Chinese medicine with less toxic side effects has become the preferred or auxiliary therapy.

In traditional Chinese medicine, Parkinson's disease belongs to the category of “tremor syndrome”, and its symptoms have been described as early as in Huangdi Neijing (“The Medical Classic of the Yellow Emperor”). Sun Yikui, a physician in the Ming Dynasty, first named Parkinson's disease as “tremor syndrome” in Chishui Xuanzhu (“Black Pearl from Red River”), and scholars in the past Dynasties have accumulated corresponding discussions and experiences. At present, it is believed that the pathogenesis of Parkinson's disease is generally deficiency in origin and excess in superficiality, wherein deficiency in origin is deficiency of liver and kidney, deficiency of essence and blood, and excess in superficiality is wind, fire, phlegm, stasis, toxin and cold. In the Third Symposium on Senile Encephalopathy of the All-China Association of Traditional Chinese Medicine, the syndrome patterns of “tremor syndrome” was defined as phlegm-heat causing wind, blood stasis causing wind, deficiency of both qi and blood, deficiency of liver and kidney, and deficiency of both Yin and Yang.

Fructus lycii, the name of a traditional Chinese medicine, is the mature fruit of Lycium barbarum in Ningxia, which is known as a precious Chinese herbal medicine from ancient times to the present, and is used in a variety of tonic prescriptions. It is recorded in Shen Nong's Herbal Classic that “Fructus lycii governs internal pathogenic factors, causes diabetes due to heat, arthralgia and rheumatism, strengthens bones and muscles, lightens the body and keeps young, and resists cold and heat after being taken for a long time”. It is sweet and smooth in taste, and has the pharmacological effects of nourishing the kidney, moistening the lung, nourishing the liver, expelling wind, improving eyesight, and treating Yin deficiency of the liver and kidney, soreness and weakness of the waist and knees, dizziness, and light-headedness. Fructus lycii has been included in the list of medicinal and edible Chinese herbal medicines by the Ministry of Health of the People's Republic of China since 2002. The homology of medicine and food is the advantage of Fructus lycii, which has a good development prospect. Modern pharmacological results show that Fructus lycii has neuroprotective, free radical scavenging, anti-inflammatory and other effects, and is widely used in health preservation, health care and other fields.

Tian Gengyuan, a professor of Shanghai Institute of Organic Chemistry, Chinese Academy of Sciences, and his colleagues first discovered Fructus lycii glucopeptide in Fructus lycii. The study has shown that Fructus lycii glucopeptide is a glycoconjugate of polysaccharide and peptide chain covalently linked extracted from Fructus lycii, which has five components (LbGp1, LbGp2, LbGp3, LbGp4 and LbGp5). It has a clear three-dimensional structure and identification method, and its preparation includes physical centrifugation alone, purification by membrane filtration, vacuum freeze-drying, etc. Fructus lycii glucopeptide is different from Fructus lycii polysaccharide previously studied. Fructus lycii polysaccharide is the general name of a class of crude polysaccharides in Fructus lycii, which is composed of six monosaccharides, including glucose, galactose, mannose, arabinose and so on. The content of Fructus lycii polysaccharide in Fructus lycii is about 2%-5%. The Chinese Pharmacopoeia requires that the content of Fructus lycii polysaccharide in Fructus lycii should not be less than 1.8% (calculated based on glucose).

Previous studies have shown that Fructus lycii polysaccharides can play a protective role in Parkinson's disease, but there is no report on the protective effect of Fructus lycii glucopeptides on Parkinson's disease.

### SUMMARY

On such a basis, the technical problem to be solved by the present invention is to provide a use of Fructus lycii glucopeptide in the preparation of a product for protecting Parkinson's disease.

The invention provides a use of Fructus lycii glucopeptide in the preparation of a product for improving Parkinson's disease.

The improvement of Parkinson's disease described in the present invention includes the protection of Parkinson-like dyskinesia.

In the preliminary experiment, it was found that the administration of Fructus lycii glucopeptide could reverse the decreased level of tyrosine hydroxylase TH (dopaminergic neuron marker) in the midbrain of mice caused model by MPTP (1-methyl-4-phenyl-1,2,3,6-tetrahydropyridine). Hereafter, the invention adopts the behavioral data as the optimal index for the improvement of the Parkinson-like symptoms, and demonstrates that the Fructus lycii glucopeptide has a significant protective effect on the Parkinson's disease through a pole-climbing experiment, a stick rotating experiment and a CatWalk gait analysis. In the present invention, the protection of Parkinson-like dyskinesia includes the improvement of motor coordination, the improvement of agility, the improvement of balance maintenance, the improvement of limb use and the stability of movement state and gait.

The pathogenesis of the Parkinson's disease is diverse, and in the present invention, the Parkinson's disease is caused by the nerve cell damage due to the neurotoxicity simulated by a drug. In the present invention, the Parkinson's disease is the Parkinson's disease caused by a neurotoxic substance. In the embodiment of the present invention, the neurotoxic substance is 1-methyl-4-phenyl-1,2,3,6-tetrahydropyridine (MPTP).

The dosage of the Fructus lycii glucopeptide for improving the Parkinson's disease is 1-100 mg/kg. In some embodiments, the dosage is 30 mg/kg.

The preparation method of the Fructus lycii glucopeptide mainly comprises the following steps: cleaning, soaking, crushing and pulping, leaching, double-channel pulping, two-stage separation, membrane separation, single-effect concentration and freeze drying. Among others, the wolfberry seeds are separated in the step of double-channel pulping, and the wolfberry pulp is separated in the step of two-stage centrifugation, the oligosaccharide is separated in the step of membrane separation, and the product finally obtained is the Fructus lycii glucopeptide. In the invention, the Fructus lycii glucopeptide comprises: LbGp1, LbGp2, LbGp3, LbGp4 and LbGp5. The protein content in LbGp1, LbGp2, LbGp3, LbGp4 and LbGp5 was 28.87%, 9.25%, 5.19%, 10.75% and 59.87%, respectively.

In some specific embodiments, the mass ratio of LbGp1, LbGp2, LbGp3, LbGp4 and LbGp5 in the Fructus lycii glucopeptide used in the present invention is (0.01-0.02):(0.005-0.01):(0.01-0.02):(0.02-0.03):(0.01-0.02). In some embodiments, the mass ratio of LbGp1, LbGp2, LbGp3, LbGp4 and LbGp5 in the Fructus lycii glucopeptide is 0.018:0.0077:0.018:0.029:0.014.

The invention also provides a product for improving Parkinson's disease, which comprises Fructus lycii glucopeptide and pharmaceutically acceptable excipients.

In the invention, the dosage form of the product comprises tablet, pill, oral liquid, capsule, syrup, dripping pill, granule, injection, powder-injection, spray, aerosol, powder-spray or suppository.

By taking an animal model of Parkinson's disease (established by intraperitoneal administration of 1-methyl-4-phenyl-1,2,3,6-tetrahydropyridine (MPTP)) as a research object, the invention has investigated the protective effect of the Fructus lycii glucopeptide on Parkinson-like dyskinesia, and compared the effect of Fructus lycii glucopeptide with that of Fructus lycii extract (water extract) and Fructus lycii polysaccharide. The results showed that Fructus lycii glucopeptide could significantly improve the Parkinson-like dyskinesia of the model animals, while no significant protective effect was observed for the Fructus lycii extract and the Fructus lycii polysaccharide at the same dosage.

### BRIEF DESCRIPTION OF THE DRAWINGS

FIG. 1 shows that the modeling and administration methods have no significant effect on the body weight of mice;

FIG. 2 shows the effect of the drugs in each group on the pole-climbing time of mice;

FIG. 3 shows the effect of the drugs in each group on the anti-fatigue time of mice;

FIG. 4 shows the effect of the drugs in each group on the average speed in the mouse CatWalk gait analysis;

FIG. 5 shows the effect of the drugs in each group on the maximum velocity change rate in the mouse CatWalk gait analysis;

FIG. 6 shows the effect of the drugs in each group on the stride frequency in the mouse CatWalk gait analysis; and

FIG. 7 shows the effect of the drugs in each group on the step number in the mouse CatWalk gait analysis.

### DETAILED DESCRIPTION

The invention provides the use of the Fructus lycii glucopeptide, which can be realized by appropriately modifying the process parameters with reference to the content of the invention. It is specifically noted that all such substitutions and modifications as would be apparent to one skilled in the art are deemed to be included in the present invention. The methods and uses of the present invention have been described in terms of preferred embodiments, and it will be apparent to those of ordinary skill in the art that the methods and uses herein can be altered or appropriately modified and combined to realize and make use of the techniques of the present invention without departing from the content, spirit, and scope of the present invention.

The materials adopted in the invention are all common commercial products and can be purchased on the market.

The preparation of the Fructus lycii glucopeptide described herein comprises a glycoconjugate of the polysaccharide and the peptide chain covalently linked obtained by subjecting the water extract of Fructus lycii to the following extraction and separation steps: physical centrifugation alone, purification by membrane filtration, abvacuum freeze-drying and other technologies. It contains LbGp1, LbGp2, LbGp3, LbGp4 and LbGp5. The protein content in LbGp1, LbGp2, LbGp3, LbGp4 and LbGp5 was 28.87%, 9.25%, 5.19%, 10.75% and 59.87%, respectively. The mass ratio of LbGp1, LbGp2, LbGp3, LbGp4 and LbGp5 in the Fructus lycii glucopeptide was 0.018:0.0077:0.018:0.029:0.014.

The present invention will be further illustrated with reference to the following Examples:

### Example 1

### I. Experiment Materials

### 1. Animals:

SPF Kunming mice, male, 6 weeks old, weighing 20-22 g, were purchased from Guangdong Medical Laboratory Animal Center (License No. SCXK (Yue) 2018-0002). The mice were raised at the temperature of 23±2° C. and the humidity of 50±10%. They are allowed free access to food and water, with standard mouse feed.

### 2. Drugs and Reagents:

MPTP: ST1020, Shanghai Beyotime Biotechnology Co., Ltd.

Rasagiline: Shanghai Jizhi Biochemical Technology Co., Ltd. (China), 136236-51-6.

Fructus lycii extract: purchased from Xi'an Baichuan Biotechnology Co., Ltd.

Fructus lycii polysaccharide: purchased from Lanzhou Waterless Biotechnology Co., Ltd.

Fructus lycii glucopeptide: provided by Ningxia Tianren Goji Biotechnology.

### II. Experiment Methods and Results

### 1. Method of Administration and Weight Monitoring

Administration method: the mice were randomly divided into 6 groups with 6 mice in each group, i.e., the control group, the MPTP model group (intraperitoneal injection of MPTP, 30 mg/kg, established after five days of administration and two days of rest), the MPTP+Fructus lycii extract (water extraction) group (administration after modeling, 30 mg/kg, intragastric administration for two weeks), the MPTP+Fructus lycii polysaccharide group (administration after modeling, 30 mg/kg, intragastric administration for two weeks), the MPTP+Fructus lycii glucopeptide group (administration after modeling, 30 mg/kg, intragastric administration for 2 weeks), the MPTP+rasagiline group (administration after modeling, 1 mg/kg, intragastric administration for 2 weeks).

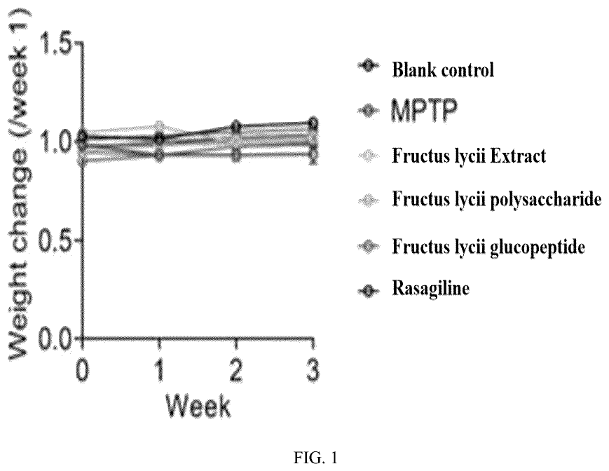

Body weight monitoring: as shown in FIG. 1, the body weight data for 3 consecutive weeks showed that the modeling and administration methods used in this experiment had no significant effect on the body weight of mice.

### 2. Methods and Results of Pole-Climbing Test

Instruction: the pole-climbing test is a behavioral test to record the time difference of mice on the pole so as to detect the limb movement coordination of mice. The whole experiment was completed in a quiet and dark environment. The pole was composed of an iron frame with a diameter of 1 cm and a height of 60 cm and a small ball with a diameter of 1.5 cm fixed on its top. Before the experiment, the surface of the pole was wrapped with gauze to prevent the mice from slipping off. Before restraint stress, mice were trained one week in advance to adapt, and each mouse was trained for five rounds every time. After restraint stress, the mice were tested once a week. During the experiment, the mice were placed on the pole with their heads upward. The time required for the mice to climb to the bottom from the beginning was recorded. If the time exceeded 1 minute, it was recorded as 1 minute. Each mouse was tested for 5 rounds each time, and the results were expressed as the average values.

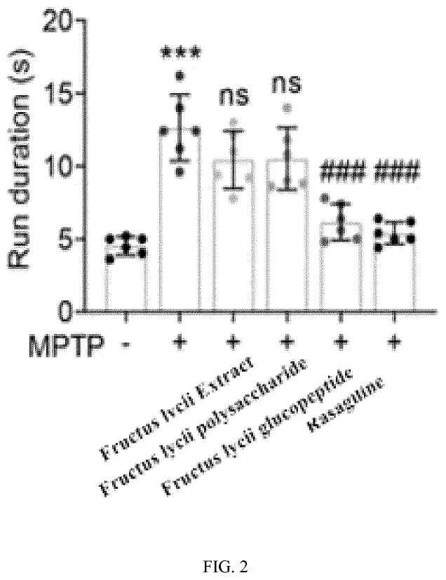

Experiment results: the time required for the mice to climb from the top to the bottom of the pole was used to evaluate the coordination ability and agility of the limbs of the mice, and the results were shown in FIG. 2. As compared with the blank control group, the time required for the mice in the MPTP group to climb the pole was significantly longer (***p<0.001 vs. blank group, n=6). Both Fructus lycii glucopeptide and the positive drug rasagiline could significantly shorten the pole-climbing time of the model mice (^{###}p<0.001 vs. the MPTP group, n=6), but Fructus lycii extract and Fructus lycii polysaccharide at the same dose as Fructus lycii glucopeptide had no significant protective effect (ns=not significant vs. the MPTP group, n=6).

### 3. Methods and Results of the Rotarod Test

Instruction: rotarod test was used to detect the motor coordination ability of mice by recording the time of the mice on the cylindrical rotarod using the rotarod fatigue apparatus. The test was conducted in a quiet and dark environment. The rotarod fatigue apparatus can accommodate five mice at a time for the test. Each mouse has a separate track, with a connected computer software for recording the time when the mouse falls. When the mouse rotates passively with the rotarod for more than two consecutive circles, the timing should also be stopped. Before restraint stress, the mice were trained for a week in advance to adapt, and each mouse was trained for three rounds every time. After restraint stress, the mice were detected once a week, and the parameter was set at a speed ramp from 5 r/min to 30 r/min at a constant rate within 5 min. The time of the mice on the rotarod within 5 min was recorded, and the time exceeding 5 min was recorded as 5 min. Each mouse was detected for 3 rounds each time, and the average value was calculated. The test interval of each group of mice was at least 30 min. Before each change of the mice, the apparatus was cleaned with 75% ethanol to remove the odor before the next group of test.

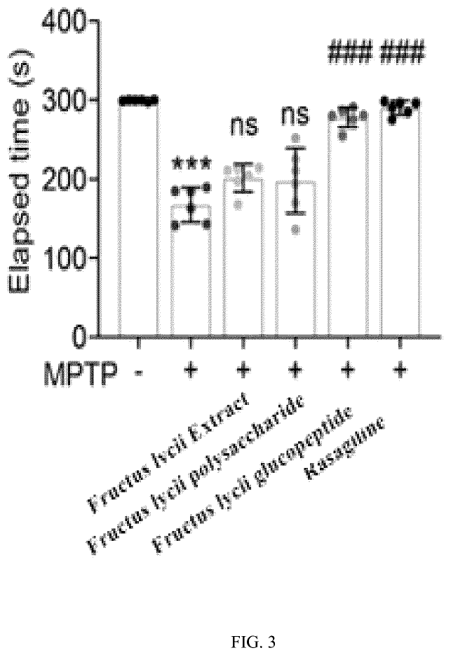

Experiment results: the motor coordination ability of the mice was evaluated by the anti-fatigue time of the mice exercising on the rotarod, and the results were shown in FIG. 3. As compared to the blank control, the on-rod time of the mice in the MPTP group was significantly shorter (***p<0.001 vs. the blank group, n=6). Both Fructus lycii glucopeptide and the positive drug rasagiline could significantly restore the on-rod time of the model mice (^{###}p<0.001 vs. the MPTP group, n=6), but Fructus lycii extract and Fructus lycii polysaccharide at the same dose as Fructus lycii glucopeptide had no significant protective effect (ns=not significant vs. the MPTP group, n=6).

### 4. Methods and Results of CatWalk Gait Analysis

Instruction: CatWalk gait analysis is a system for analyzing the gait of rodents, including the balance maintenance ability, the use of limbs and the stability analysis of movement state and gait. The main accessories include built-in red LED light panel and green fluorescent glass panel, high-frequency camera under the glass panel, image transmission system and computer software and hardware. Before inspection, the camera height will be adjusted to 60 cm, the effective length and width of the runway will be delimited in the software, and the gain of the camera will be set to 28.12 dB. The process of each mouse passing through the glass plate smoothly was recorded at room temperature, and the longest time was set to 10 s, that is, the data was valid if the mouse passed through the set distance within 10 s. The principle of recording is that the paw prints of mice are illuminated by the fluorescent reflection of the fluorescent lamp in the glass panel, captured by the camera below and transmitted to the computer, and the recorded video data are statistically analyzed by the software of the system. According to the location of paw print, the pressure of paw on the glass panel, the distance between different paw prints, the velocity of mouse gait and the regularity of gait, the gait of mice was qualitatively and quantitatively analyzed.

Before the formal test, the mice were trained three times in advance, and then the test was started, and each mouse was recorded for at least five times. The experiment should be carried out in a dark and quiet environment without human interference. After the test of each mouse, the runway should be cleaned and wiped with 75% ethanol to avoid interference with the next mouse. At the end of the test, the resulting data were statistically analyzed by Noldus CatWalk XT 9.1 system.

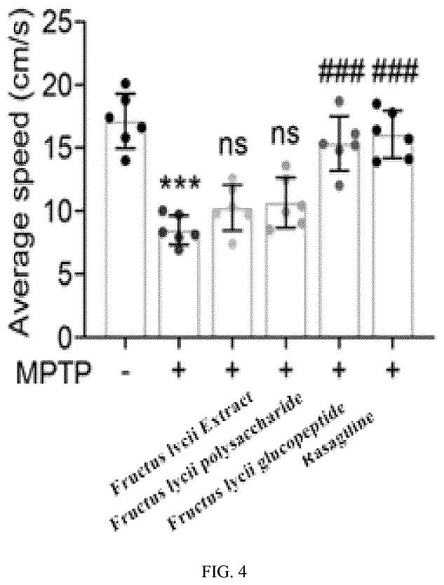

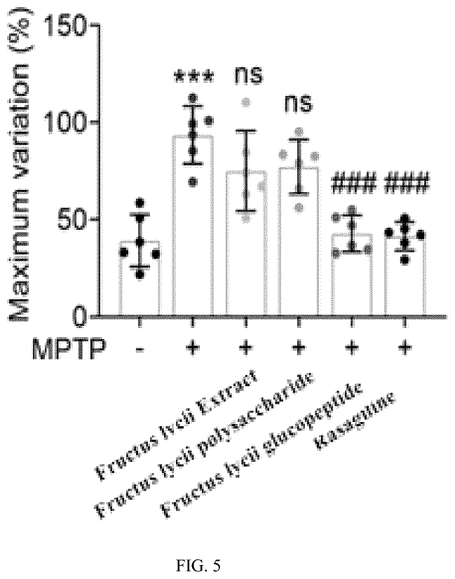

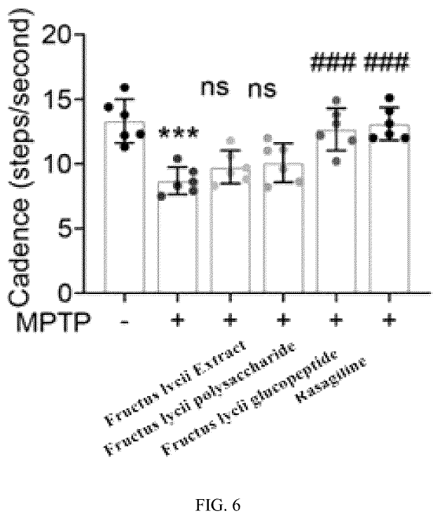

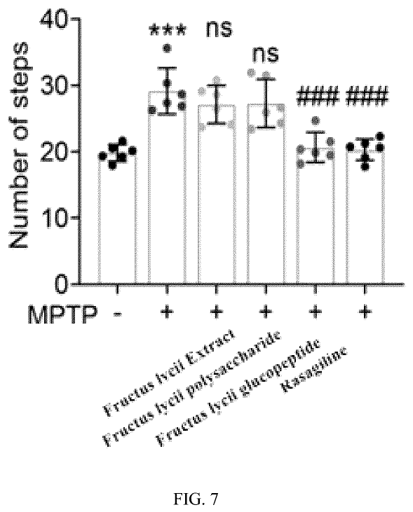

Experiment results: the gait changes caused by motor defects and other reasons were evaluated by comprehensively analyzing the movement trajectory of mice during natural walking, and the results were shown in FIG. 4-7. The evaluation indexes used include average speed, maximum variation, cadence and number of steps. As compared with the blank control group, the mice in the MPTP group showed significant damage in the above evaluation indexes (***p<0.001 vs. the blank group, n=6). Both Fructus lycii glucopeptide and the positive drug rasagiline could significantly restore the gait behavior disorder in model mice (^{###}p<0.001 vs. the MPTP group, n=6), but Fructus lycii extract and Fructus lycii polysaccharide at the same dose as Fructus lycii glucopeptide had no significant protective effect (ns=not significant vs. the MPTP group, n=6).

The above described is only the preferable embodiments of the present invention, and it should be noted that several improvements and modifications can be made by the person of ordinary skill in the art without departing from the principles of the present invention. These improvements and modifications should also be regarded as in the scope of the present invention.

1. Use of Fructus lycii glucopeptide in the preparation of a product for improving Parkinson's disease.

2. The use according to claim 1, wherein the improvement includes the protection of Parkinson-like dyskinesia.

3. The use according to claim 2, wherein the protection of Parkinson-like dyskinesia includes the improvement of motor coordination, the improvement of agility, the improvement of balance maintenance, the improvement of limb use and the stability of movement state and gait.

4. The use according to claim 1, wherein the Parkinson's disease is the Parkinson's disease caused by a neurotoxic substance.

5. The use according to claim 4, wherein the neurotoxic substance is 1-methyl-4-phenyl-1,2,3,6-tetrahydropyridine.

6. The use according to claim 1, wherein the Fructus lycii glucopeptide comprises: LbGp1, LbGp2, LbGp3, LbGp4 and LbGp5.

7. The use according to claim 6, wherein, in the Fructus lycii glucopeptide, the mass ratio of LbGp1, LbGp2, LbGp3, LbGp4 and LbGp5 is (0.01-0.02):(0.005-0.01):(0.01-0.02):(0.02-0.03):(0.01-0.02).

8. The use according to claim 1, wherein the dosage of the Fructus lycii glucopeptide for improving the Parkinson's disease is 1-100 mg/kg.

9. A product for improving Parkinson's disease, comprising Fructus lycii glucopeptide and pharmaceutically acceptable excipients.

10. The product according to claim 9, wherein the dosage form of the product comprises: tablet, pill, oral liquid, capsule, syrup, dripping pill, granule, injection, powder-injection, spray, aerosol, powder-spray or suppository.
